(19) United States
(12) Patent Application Publication
(10) Pub. No.: US 2025/0322215 A1
(43) Pub. Date: Oct. 16, 2025

(54) DESIGN METHOD OF FUNCTIONAL PROTEINS USING PROTEIN LARGE LANGUAGE MODEL FINE-TUNING TECHNOLOGY

(71) Applicant: The Chinese University of Hong Kong, Hong Kong (CN)

(72) Inventors: Pheng Ann HENG, Hong Kong (CN); Guangyong CHEN, Hangzhou (CN); Hanqun CAO, Hong Kong (CN)

(21) Appl. No.: 18/636,388

(22) Filed: Apr. 16, 2024

(51) Int. Cl. G06N 3/0475; G06N 3/045; G06N 3/096; G16B 15/00; G16B 40/20

(52) U.S. Cl. CPC G06N 3/0475; G06N 3/045; G06N 3/096; G16B 15/00; G16B 40/20

(57) ABSTRACT

A method for functional antimicrobial peptide design is provided. The method includes running pretrained protein large language models as a generator and enhancing a sample candidate. The enhancing a sample candidate includes performing an automatic pipeline based on machine learning methods and a plurality of bioinformatics methods and is configured to perform protein inverse folding and computational protein sequence designing. The computational protein sequence designing includes running a pretrained deep learning-based protein structure model, an autoregressive pretrained protein sequence model, and a deep learning-based alignment model. The pretrained deep learning-based protein structure model is configured to learn three-dimensional structures of the proteins and output latent structure embeddings in a high dimensional space. The autoregressive pretrained protein sequence model includes a protein language model to automatically generate protein sequences and is trained to predict next amino acid in a protein sequence based on a preceding sequence(s) of amino acids.

This invention is made with government support under Hong Kong ITF grant number ITS/241/21. The government has certain rights in the invention.

### BACKGROUND OF THE INVENTION

Antimicrobial Resistance (AMR) has become a significant concern in global human health. Its unique advantage lies in its ability to evolve, enabling pathogens to resist the effects of drugs that are once effective against them, and its potential to spread, posing a threat to public health on a global scale [1, 2]. This has made the development of new antimicrobial strategies a pressing necessity. Antimicrobial peptides (AMPs) play a crucial role in this context [3, 4]. Their importance stems from their broad-spectrum activity against a wide range of pathogens, including those resistant to traditional antibiotics. Furthermore, AMPs operate through multiple mechanisms of action, making it difficult for pathogens to develop resistance against them. This makes AMPs a promising alternative in the fight against AMR [5, 6].

The conventional methods for discovering AMPs involve isolating and purifying samples from natural sources, followed by extensive screening to identify promising candidates [7, 8]. These approaches have yielded valuable insights into the structural and functional characteristics of AMPs and their antimicrobial mechanisms. Nonetheless, the manual curation and resource-intensive nature of these methods impede the efficient exploration of the vast landscape of AMPs. However, traditional AMP discovery overlooks rare or elusive AMPS with untapped antimicrobial potential, which prevents researchers from inadvertently excluding novel AMPs present in unexplored ecological niches. Furthermore, the time-consuming isolation and purification steps hinder the rapid identification of AMPs, impeding their translation into clinical applications [9]. Novel approaches addressing the inefficiencies of traditional methods are required to facilitate the identification and characterization of AMPs more efficiently and comprehensively.

In recent years, the application of AI algorithms, particularly machine learning and deep learning models, has emerged as a promising approach to enhance the pre-diction, design, and optimization of antimicrobial peptides (AMPs) [10, 11]. These models, trained on experimentally validated AMP datasets, offer significant advantages over traditional methods, and provide valuable insights into the antimicrobial potential of candidate peptides. By considering various features, such as amino acid composition and secondary structure, and hydrophobicity, AI models enables accurate classification and prediction of candidate peptides antimicrobial activity [12]. This allows researchers to prioritize the most promising candidates for further experimental validation, streamlining the discovery process and saving valuable time and resources. AI-based approaches explore vast spaces of potential peptide candidates and analyze larger dataset, serving as powerful assistants for human experts. Moreover, AI methods offer a more comprehensive understanding of the mode of action and spectrum of antimicrobial activity exhibited by AMPs. By training models on experimentally validated AMP datasets, these algorithms learn patterns and relationships between peptide features and their functional properties [13]. This knowledge can then be used to predict the antimicrobial mechanisms employed by candidate peptides, enabling researchers to select peptides with specific modes of action tailored to combat specific pathogens.

In addition to prediction tasks, AI-based approaches facilitate the design and optimization of AMPs with enhanced efficacy and stability. Computational tools assist in rational peptide design by predicting and optimizing peptide structures with desirable physicochemical properties. This includes modifying peptide sequences to improve antimicrobial activity, selectivity, and resistance against degradation [14]. Through virtual screening and molecular dynamics simulations, researchers can investigate the interactions between AMPs and microbial targets, providing valuable insights for the development of AMP-based therapeutics [15-17]. Despite the advantages of AI and bioinformatics, integration with experimental validation remains crucial. Identified candidate peptides must undergo rigorous testing using in vitro and in vivo assays to validate their antimicrobial activity, cytotoxicity, and pharmacokinetic properties, resulting in lengthy and costly experimental validation cycles. By combining the strengths of computational approaches with experimental validation, researchers can overcome the limitations of traditional methods, accelerating the discovery and development of novel AMPs to combat antimicrobial resistance [18, 19].

Despite these advances, the generation of novel AMPs with specific desired properties remains a daunting task, as deep learning models primarily rely on the patterns and information present in the training data. Consequently, the quest to create entirely new and effective AMPs with tailored characteristics continues to be an active area of ongoing research [19]. In situations where the training dataset is limited, biased, or incomplete, deep learning models may struggle to generalize effectively, leading to sub-optimal results. This emphasizes the necessity for a more robust approach that takes into account these limitations and can deliver enhanced performance and accuracy.

To overcome these challenges, researchers are actively exploring hybrid approaches that combine the strengths of deep learning with other computational techniques, including molecular dynamics simulations and evolutionary algorithms. By integrating these complementary methodologies, it becomes possible to leverage the power of deep learning for pattern recognition while also incorporating the ability to explore the vast space of novel AMP sequences [11]. Besides, multi-stage generative modeling methods based on semi-supervised learning pattern also holds promising potential to address this problem. Several successful approaches based on deep generative models have been developed to explore functional antimicrobial peptides (AMPs) by employing conditional sampling with specific functional labels. Due to the limited availability of labeled training data, these methods are trained in a semi-supervised manner [20, 21]. GM-Pep trains a vanilla VAE using unlabeled peptides and an additional classifier using a small-scale dataset [22]. The generated samples are then filtered using the trained multi-classifier. PepCVAE utilizes 1.7 million unlabeled data from UniProt (Geneva, Switzerland) [23] for vanilla self-supervised training, along with 1,500 labeled AMP data for guiding conditional sampling in the VAE [24]. AMPGANv2 conducts conditional sampling based on GAN, using a combined dataset of 6,550 AMPs and 49,000 non-AMPs for joint training, where the objective function includes log loss for classification and MSE loss for self-recovery [25]. DeepImmuno explores T-cell immunity peptides using a similar framework to AMPGANv2 [26]. HydrAMP extends the conditional VAE with MAP by incorporating high MIC value [19]. The training data comprises 3,444 peptides with high MIC value, 22,000 AMPs, and about 220,000 unlabeled sequences from open-source database, resulting in more detailed and efficient generation.

Despite the immense potential of the generative method, current semi-supervised based generative approaches explore only limited potential of large scale of unlabeled data. This constraint significantly impacts their performance and restricts the scope of exploration. Moreover, these models are susceptible to various shortcomings that hinder their effectiveness, such as unstable training dynamics, the need for two-stage training procedures, intricate filtering mechanisms, and limited generalization capabilities. These issues underscore the urgent need for advancements in this domain [18, 19].

On the other hand, the Protein Large Language Model (pLM) represents a promising approach to AMP discovery, characterized by its expansive exploration space, diverse generation capabilities, and novelty [27, 28]. However, the pLM faces challenges, including high computational cost, difficulties in generating short proteins (12-50AA), and instability in conditional generation. Addressing these limitations is crucial to fully harness the capabilities of the pLM and advance the field of AMP discovery [29, 30]. Thus, novel strategies are required to effectively address the needs of the industry.

### BRIEF SUMMARY OF THE INVENTION

There continues to be a need in the art for improved designs and techniques for a method and systems for a design method of functional proteins using protein large language model with fine-tuning technology.

According to an embodiment of the subject invention, a method based on deep learning-based framework for functional antimicrobial peptide design is provided, comprising running pretrained protein large language models as a generator and enhancing a sample candidate. The enhancing a sample candidate comprises performing an automatic pipeline based on machine learning methods and a plurality of bioinformatics methods. The automatic pipeline is configured to perform protein inverse folding and computational protein sequence designing. The computational protein sequence designing comprises running a pretrained deep learning-based protein structure model; running an autoregressive pretrained protein sequence model; and running a deep learning-based alignment model. The pretrained deep learning-based protein structure model comprises a plurality of data. Moreover, the pretrained deep learning-based protein structure model is configured to learn three-dimensional structures of the proteins. In addition, the pretrained deep learning-based protein structure model is configured to output latent structure embeddings in a high dimensional space. The autoregressive pretrained protein sequence model comprises a protein language model to automatically generate protein sequences. The autoregressive pretrained protein sequence model is trained to predict next amino acid in a protein sequence based on a preceding sequence(s) of amino acids. The deep learning-based alignment model is trained to learn connections between latent representations of protein sequences and protein structures.

In certain embodiment of the subject invention, a method based on deep learning-based framework for functional antimicrobial peptide design is provided, the comprising an adapter-based step and a candidate selection step. The adapter-based step comprises a supervised training performed on a plurality of peptide families. The adapter-based step further comprises a feedback tuning step configuring the pretrained protein language model for special property generation. The special property generation includes one, any or all of hydrophobicity, electric charge, cell toxicity, and hemolytic activity. The candidate selection step comprises a filtering step and an analyzing step. Moreover, the candidate selection step comprises applying sequence embeddings from pretrained protein sequence encoders into a k-nearest neighbors (K-NN) search process and a MIC identification process. The candidate selection step further comprises selecting an intersection of two potential subsets respectively generated from the K-NN search process and the MIC identification process for post-processing. The candidate selection step may additionally comprise performing a folding method, a sequence similarity search, and a structure alignment.

### BRIEF DESCRIPTION OF THE DRAWINGS

FIG. 1 shows a conditional generation pipeline for AMP design, wherein the training and finetuning stages comprise an adapter-based step and a candidate selection step, wherein the adapter-based step comprises supervised training on multiple peptide families, wherein the feedback fine-tuning is configured to instruct pretrained protein language model for special property generation, including hydrophobicity, electric charge, cell toxicity, and hemolytic activity; wherein the candidate selection step comprises a filtering step and an analyzing step, wherein sequence embeddings are applied from pretrained protein sequence encoders into K-NN search and MIC identification process, then, the intersection of two potential subsets is selected for post-processing, and finally, bioinformatics tools comprising a folding algorithm, a sequence similarity search, and a structure alignment, are employed for further analysis, according to an embodiment of the subject invention.

### DETAILED DISCLOSURE OF THE INVENTION

A deep learning-based framework for functional antimicrobial peptide design is provided. Compared to the previous deep learning approaches, the methods of the subject invention apply pretrained protein large language models as the generator, achieving high-quality generation with the combination of fine-tuning techniques. Furthermore, the sampled candidates are enhanced by an automatic pipeline based on machine learning algorithms and bioinformatics tools. In the experiments of the subject invention, antimicrobial peptides with multiple activities to various types of strains are successfully designed, together with multiple desired biochemistry properties. It is expected that the comprehensive framework based on the protein large language models could provide a new perspective for functional AMP generation, even for function protein sequence generation.

### Selected Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The term “about” means within an acceptable error range for the particular value as determined by one of ordinary skill in the art, which depends in part on how the value is measured, i.e., the limitations of the measurement system. In the context of compositions comprising amounts of ingredients where the term “about” is used, these compositions comprise the stated amount of the ingredient with a variation (error range) of 0-10% around the value (X±10%). In other contexts, the term “about” is used provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids comprising known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

In this application, the terms “polypeptide”, “peptide”, and “protein” are used interchangeably herein to refer to a polymer of amino acids. The terms apply to amino acid polymers in which one or more amino acid residues are artificial chemical mimetic of a corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used herein, “autoencoder” refers to a type of artificial neural network used to efficiently code unlabeled data.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, “peptide design” and “functional protein design” are used interchangeably to refer to a process of identifying protein sequences.

As used herein, “deep learning” refers to a subset of machine learning methods based on artificial neural networks with representation learning.

As used herein, “large language model”, “LLM”, and “LLMs” are used interchangeably to refer to a language model that is able to achieve general-purpose language generation.

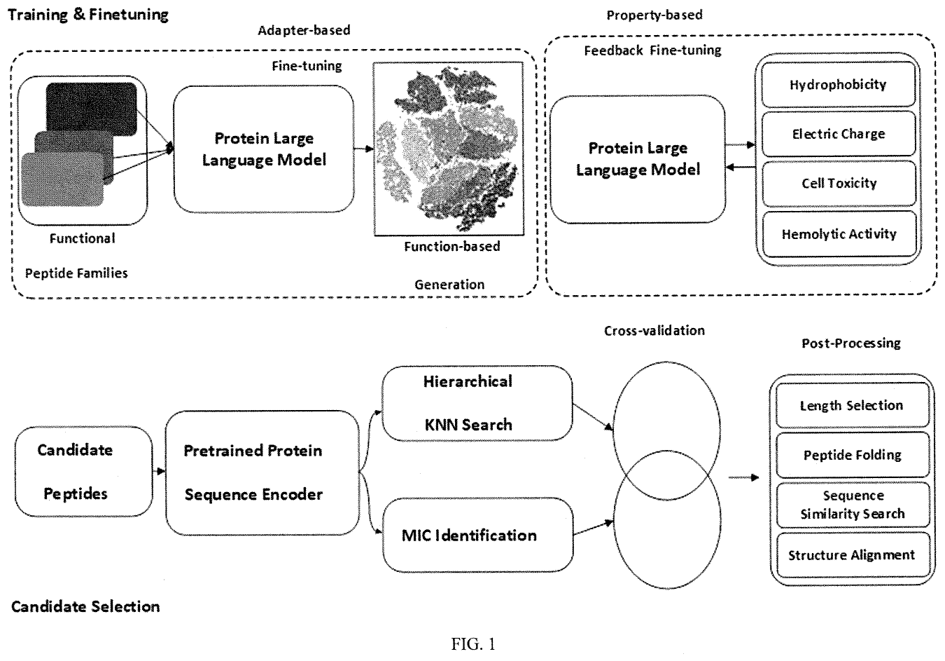

Referring to FIG. 1, a conditional generation pipeline for AMP design is shown. The training and finetuning stages comprise two steps. First, the adapter-based step includes supervised training on multiple peptide families. The feedback tuning aims to instruct pretrained protein language model for special property generation, including hydrophobicity, electric charge, cell toxicity, and hemolytic activity. The candidate selection step comprises a filtering step and an analyzing step for post-processing. The filtering step comprises applying sequence embeddings from pretrained protein sequence encoders into K-NN search and MIC identification process and selecting the intersection of two potential subsets for the post-processing. Finally, in the analyzing step for post-processing, bioinformatics tools are employed for further analysis, which comprises a length selection method, a folding method, a sequence similarity search method, and a structure alignment method, according to an embodiment of the subject invention.

In one embodiment, the folding method is a Peptide Folding method.

In one embodiment, the sequence similarity search method is a BLAST search method.

In one embodiment, the structure alignment method is a Foldseek Search method.

### Protein Sequencing

In some embodiments, the pipeline of the subject invention is designed for protein inverse folding and computational protein sequence design. It is a sophisticated system comprises several components, each of which plays a significant role in the overall process. The first component is a pretrained deep learning-based protein structure model. This model is trained on a vast amount of data and is capable of learning the three-dimensional structures of proteins. It is designed to output latent structure embeddings in a high dimensional space. This means that the model can generate a compressed representation of a protein's structure, which can then be used for further analysis or to feed into other models. The second component of the pipeline is an autoregressive pretrained protein sequence model. This model leverages protein language modeling techniques to generate protein sequences automatically. Autoregressive models are powerful tools in machine learning that are capable of predicting a sequence of outputs based on a sequence of inputs. In some embodiments, the model is trained to predict the next amino acid in a protein sequence based on the preceding sequence of amino acids. The third component is a deep learning-based alignment model. This model is trained to learn the connections between the latent representations of protein sequences and protein structures. The alignment model can be a crucial part of the pipeline as it bridges the gap between the sequence and structure of proteins. It does this by learning to map the latent representations of protein sequences to their corresponding protein structures. This is a challenging task due to the complex nature of proteins, but a successful alignment model can provide valuable insights into the relationship between a protein's sequence and its structure. In summary, the pipeline offers a comprehensive solution for protein inverse folding and computational protein sequence design. It leverages advanced deep learning techniques to learn protein structures, generate protein sequences, and align these sequences with their corresponding structures.

### Methods of Use

The subject invention can have multiple uses in different industries. In some embodiments, the subject invention can be used in the pharmaceutical, medical, agriculture, biotechnology/energy, food/beverage and environmental monitoring. Functional protein synthesis has significant applications in the pharmaceutical and medical fields. The end-to-end deep learning pipeline can expedite the drug development process, reducing time and costs. It can be used for predicting protein structures and interactions, optimizing drug design, and accelerating drug screening and discovery. In the agricultural sector, the deep learning pipeline for functional protein synthesis can be applied in agriculture to enhance crop yield and quality. By predicting and optimizing the functionality of crop proteins, it is possible to improve disease resistance, drought tolerance, salt tolerance, and other characteristics, thereby enhancing crop adaptability and productivity. In the biotechnology and energy sector, the end-to-end deep learning pipeline also has extensive potential applications in biotechnology and the energy sector. It can be used for designing and optimizing biocatalysts to improve the efficiency of biofuel production and other biotechnological processes. Additionally, it can aid in the development of new biomaterials and bioenergy technologies. In the food and beverage industry, the application of functional proteins can also extend to the food and beverage industry. Through the deep learning pipeline, it is possible to predict and enhance the functionality and taste of proteins in food and beverages, thereby developing products with improved nutritional value and flavor. In environmental monitoring and pollution control, the end-to-end deep learning pipeline can be used for monitoring proteins in the environment, such as microbial proteins in water. In some embodiments, this is crucial for environmental pollution control and water quality management.

### Materials and Methods

### Diverse Peptide Sampling

To achieve high-quality and diverse peptide sampling over a wider protein distribution, auto-regressive pretrained protein large language model ProGen2 is employed as the baseline model for functional generation. The framework includes two main stages, including efficient fine-tuning and multi-level filtering. To fully explore the wide distribution of protein sequences, fine-tuned ProGen2 [29] models are expected to search antimicrobial peptides over the protein universe instead of the empirical distribution of small-scale training data. Moreover, online reinforcement learning based fine-tuning technique is employed to enable protein language model to achieve functional generation with specific molecule-based properties such as hydrophobicity and electric charge [16]. In the second stage, to further improve generation quality, a pipeline based on machine learning algorithm as well as bioinformatics analysis is provided. The cross-validation within different filtering and analysis processes, candidate sequences are required to have multiple desired properties and higher similarity to antimicrobial peptide distribution. Moreover, the bioinformatics analysis ensures flexible and efficient biosynthesis and new possibilities for discovery of new antibacterial mechanisms.

### Data Preparation

To achieve full exploration of protein language model's generative power, a comprehensive dataset with as many as possible instances with multiple desired functions is constructed. The data are fetched from multiple open-source AMP database (AMP Technologies, Mountain View, USA), including DRAMP [33], DADP [34], LAMP2 [35], dbAMPv2.0 [36], and AMPScanner [37].

AMPs with reported antimicrobial, antibacterial, and antifungal activities are collected. Also, the AMP data with reported MIC values, which results in a similar MIC dataset to HydrAMP [19] are collected. Then, the data are filtered according to length [symbol] 100 and instances with redundancy. The dataset comprises 27,148 instances, being split into training, validation, and testing datasets with ratio 8:1:1. To facilitate the highly efficient finetuning on multiple peptide families, functional labels for all collected AMPs are divided into sub-families according to functional labels. The processed dataset is applied to the following fine-tuning stages as well as the machine learning-based filtering steps, aiming to present full exploration to AMP natural distribution.

### Efficient Fine-Tuning

Compared to conditional generation methods based on variational auto-encoders and generative adversarial networks, protein large language models offer enhanced robust-ness in generating protein sequence embeddings that capture specific distributions, while also facilitating more accurate global and local interactions among amino acids. In this study, ProGen2 is utilized as the baseline generation model, which shares simi-lar network architectures with Generative Pre-trained Transformer (GPT) models [38]. These architectures comprise attention-based language blocks and are initialized with pretrained weights, keeping the network structures fixed. Moreover, the auto-regressive pattern employed in generation provides greater flexibility in terms of sequence length and sample diversity, thereby increasing the potential for generating previously unseen novel protein sequences with desired properties.

However, the training and inference processes for these pretrained large language models come with higher computational burdens. To mitigate this issue, a fine-tuning method involving two sub-stages that employ different efficient fine-tuning techniques is provided. Specifically, LoRA (LoRa Alliance, San Ramon, USA) [31], an adapter-based fine-tuning approach, which achieves efficient tuning with a small number of parameters is adopted. This approach not only addresses the computational burden but also helps prevent model overfitting when training with small-scale datasets. Rank-based adapters are introduced into the attention blocks of ProGen2 to regulate residue interactions for conditional generation. The fine-tuning of the model is guided by perplexity, which represents the expectation of empirical cross-entropy between previous tokens and the next-generated tokens. By fine-tuning the models, a series of possible peptides with similar functions are generated to the training data. Furthermore, the generation process is optimized based on specific properties such as hydrophobicity and electric charge to facilitate their application in biological contexts.

Hydrophobicity plays a vital role in facilitating the interaction and disruption of lipid-rich microbial cell membranes by antimicrobial peptides (AMPs). AMPs readily integrate into the lipid bilayer, resulting in membrane permeabilization and eventual cell demise [39]. The positive charge of AMPs facilitates their interaction with negatively charged surfaces of microbial cells, including bacterial membrane components, promoting binding and compromising membrane integrity [8]. To access these valuable properties through a filtering process without requiring feedback, an online reinforcement learning technique based on the properties of the generated sequences is applied. Inspired by RLHF [32], a reward function that utilizes the out-put peptide prediction scores from identification software is designed. By connecting this reward function to the measurable loss of ProGen2, preference data are further generated by manipulating the candidate space for generation.

In summary, it is demonstrated that the advantages of the protein large language models of the subject invention is superior to the existing conditional generation methods. A two-stage fine-tuning method with efficient techniques is adopted to address the computational burden associated with training and inference. Through the regulation of residue interactions and optimization based on specific properties, peptides with desired functions for potential biological applications are generated. Additionally, by employing online reinforcement learning techniques, the generated sequences can be filtered and optimized based on valuable properties, such as hydrophobicity and electric charge. Overall, the approach provides a promising avenue for advancing protein sequence generation and its application in various domains.

### Comprehensive Filtering and Analysis

To enhance the quality of the generated candidates from the fine-tuned models and improve the feasibility of biosynthesis, a pipeline for function-based peptide filtering and bioinformatics analysis is provided. This pipeline comprise machine learning-based methods for obtaining candidates with high functional similarity to the target peptide sequence distributions. A pre-processing stage based on bioinformatics tools is also incorporated to provide more valuable candidates for biological verification experiments.

### Machine Learning Based Filtering

Given the limited amount of training data, the fine-tuned models may not generate samples that perfectly fit the desired distribution. Therefore, a filtering stage is introduced to select candidates with a higher likelihood of being functional antimicrobial peptides (AMPs) [40]. Initially, the candidates are projected into a wide protein latent space using the bi-directional pretrained protein large language models ESM2 (ESM II Inc, Amherst, USA) [27]. These models, trained on millions of protein sequences, provide robust sequence embeddings capable of capturing hidden relationships among different candidates. Similar to BERT (BioRxiv, New York, USA) [41], a dominant Transformer-based architecture in natural language processing, ESM2 is trained using masked language modeling. For the peptide candidates, the ESM2-650M is utilized as the base protein sequence encoder, with its pretrained weights fixed.

Based on the assumption that proteins with similar biological functions exhibit similar sequences, the embedding-based filtering process leverages the robust embed-dings to capture the hidden connections among functional protein sequences. Then, the similarity between the generated candidates and the target functional sequences are measured using a series of machine learning-based approaches, including K-Nearest Neighbor (KNN) and Bayesian classification based on the minimum inhibitory concentration (MIC) activity of AMPs [19].

### KNN-Based Multi-View Filtering

For the KNN-based filtering, a K-nearest neighbors machine learning method is employed to effectively filter the generated antimicrobial peptide samples. To assess the similarity between the generated samples and the fine-tuning data samples, the distances between their respective protein sequence embeddings obtained from ESM2 [27] are computed. A greater similarity between the samples indicates a higher likelihood of possessing similar properties. To establish a filtering criterion, the weighted average of distances between the sequence embeddings of the generated samples and the centers of the target functional sample sequence embeddings are computed. This criterion serves as the basis for the subsequent filtering process. Additionally, multiple metrics for latent distances are incorporated to assess similarity from different perspectives, including Euclidean Distance, Manhattan Distance, and cosine similarity. By considering diverse metrics, the approach comprehensively evaluates various aspects, mitigates the influence of outliers, and improves the overall accuracy and flexibility of the algorithm. To address the issue of different metric scales spanning distinct ranges, normalization on the three metrics is performed. This normalization process ensures that the metrics are brought to a comparable range. Subsequently, the normalized metrics are summed together to yield final similarity scores, facilitating a more comprehensive assessment of similarity.

### Bayesian-Inspired MIC Filtering

To further refine the selection of functional AMP candidates, a Bayesian classifier is incorporated into the pipeline. This classifier utilizes the minimum inhibitory concentration (MIC) activity of AMPs as a criterion for classification. In this process, existing training data that includes specific MIC values associated with antimicrobial peptides are leveraged.

To train the classifier, the ESM embeddings of the antimicrobial peptide sequences are utilized as inputs. These embeddings capture the essential features of the peptides and provide a compact representation for further analysis. The classifier is designed to predict the likelihood of a candidate peptide possessing antimicrobial activity based on its ESM embedding. The classifier is trained using a labeled dataset, where each sample includes the ESM embedding of a peptide and its corresponding MIC value. The training objective is to minimize the L1 loss between the predicted values of the classifier and the true MIC values.

Once the classifier is trained, it serves as an additional filtering tool in the pipeline. During the filtering process, the pretrained weights of the classifier are utilized and an offset activity threshold is set. Candidates with predicted MIC values above this threshold are considered to have high antimicrobial activity and are retained, while those below the threshold are filtered out. This approach allows us to prioritize candidates that are more likely to exhibit the desired antimicrobial properties, saving time and resources in the subsequent stages of the analysis and experimentation.

### Cross Validation on Candidates

In order to enhance the credibility and success rate of machine learning algorithms, a highly refined approach is adopted. The objective is to ensure that the selected samples not only maintain similarity to functional antimicrobial peptides but also exhibit relatively satisfactory activity. To accomplish this goal, a cross-validation technique that combines subsets of samples identified through KNN method and classifier method is employed, utilizing their intersection as the post-processing candidate set.

This cross-validation algorithm harnesses the strengths of each individual algorithm, yielding a more reliable and consistent candidate set. Moreover, the convergence of different metrics reduces inherent biases associated with each algorithm. Ultimately, the selection of candidates that simultaneously satisfy multiple criteria enhances the robustness and generalizability of the screening process.

### Post-Processing

Post-processing serves as a critical step in the refinement and analysis of antimicrobial peptide screening, ensuring the reliability and relevance of the selected sequences. By harnessing a range of advanced bioinformatics tools, valuable insights are extracted and the outcomes are aligned with practical applications in the laboratory. Serving as a reference for experimental validation, post-processing aims to uncover novel antimicrobial mechanisms that can combat the growing threat of drug-resistant microorganisms.

### Length Selection

In the post-processing pipeline, length selection plays a pivotal role. the peptide length is carefully determined, being restricted to 50 amino acids or fewer [18]. This constraint facilitates the synthesis process, making it more feasible and efficient for experimental validation. By focusing on shorter peptides, their compatibility with laboratory-scale synthesis techniques is optimized while their potential for effective antimicrobial activity is maintained.

### Structure Folding

To gain a deeper understanding of the structural properties of the selected peptides, state-of-the-art protein folding tools, including AlphaFold2 (Alphafold, Hixton, UK) [42] and ESM-Fold [43] are employed. Leveraging the power of deep learning and artificial intelligence, these tools provide invaluable insights into the foldability of peptides and their thermal stability. By simulating the folding process and analyzing the resulting structures, the stability and conformational characteristics of the peptides can be predicted, guiding the design of peptides with improved structural integrity and functional properties.

### Sequence Alignment Via BLAST Scoring

Furthermore, the post-processing method incorporates the widely used BLAST sequence alignment tool (BLAST, Newport Beach, USA) [44]. This allows us to assess the novelty of the selected candidates by comparing them to existing peptide sequences. Additionally, their similarity to known functional domains is investigated, allowing for identifying promising candidates that may exhibit similar activity or belong to specific functional families. This comparative analysis enhances the understanding of the potential mechanisms of action and functional properties of the selected peptides, providing valuable insights for further experimental investigations.

### Structure Search by FoldSeek

In addition to BLAST, the power of FoldSeek (Foldseek Search) [45], an efficient structure search tool, is leveraged. By employing FoldSeek, structurally similar peptide families and uncover potential functional domains that may contribute to the antimicrobial activity of the selected candidates are identified. This exploration of structural motifs and functional domains opens up new avenues for investigating the diverse mechanisms by which antimicrobial peptides exert their activity, facilitating the discovery of innovative strategies to combat antibiotic resistance.

The holistic post-processing methodology is a combination of length selection, protein folding analysis, BLAST sequence alignment, and FoldSeek, enabling a meticulous examination of the chosen antimicrobial peptides. By seamlessly integrating bioinformatics analysis with laboratory synthesis and validation, profound insights into their structural attributes, functional traits, and prospective mechanisms of action are gained. The synergistic approach holds immense potential for the advancement of groundbreaking antimicrobial therapies, offering a promising solution to combat the pressing challenge of drug-resistant pathogens.

Embodiment 1. A method based on deep learning-based framework for functional antimicrobial peptide design, the method comprising:
running pretrained protein large language models as a generator; and
enhancing a sample candidate.

Embodiment 2. The method of embodiment 1, wherein the enhancing a sample candidate comprises performing an automatic pipeline based on machine learning methods and a plurality of bioinformatics methods.

Embodiment 3. The method of embodiment 2, wherein the automatic pipeline is configured to perform protein inverse folding and computational protein sequence designing.

Embodiment 4. The method of embodiment 3, wherein the computational protein sequence designing comprises:
running a pretrained deep learning-based protein structure model;
running an autoregressive pretrained protein sequence model; and
running a deep learning-based alignment model.

Embodiment 5. The method of embodiment 4, wherein the pretrained deep learning-based protein structure model comprises a plurality of data.

Embodiment 6. The method of embodiment 4, wherein the pretrained deep learning-based protein structure model is configured to learn three-dimensional structures of the proteins.

Embodiment 7. The method of embodiment 4, wherein the pretrained deep learning-based protein structure model is configured to output latent structure embeddings in a high dimensional space.

Embodiment 8. The method of embodiment 4, wherein the autoregressive pretrained protein sequence model comprises a protein language model to automatically generate protein sequences.

Embodiment 9. The method of embodiment 8, wherein the autoregressive pretrained protein sequence model is trained to predict next amino acid in a protein sequence based on a preceding sequence(s) of amino acids.

Embodiment 10. The method of embodiment 4, wherein the deep learning-based alignment model is trained to learn connections between latent representations of protein sequences and protein structures.

Embodiment 11. A method based on deep learning-based framework for functional antimicrobial peptide design, the method comprising:
an adapter-based step; and
a candidate selection step.

Embodiment 12. The method of embodiment 11, wherein the adapter-based step comprises a supervised training performed on a plurality of peptide families.

Embodiment 13. The method of embodiment 12, wherein the adapter-based step further comprises a feedback tuning step configuring the pretrained protein language model for special property generation.

Embodiment 14. The method of embodiment 13, wherein the special property generation includes one, any or all of hydrophobicity, electric charge, cell toxicity, and hemolytic activity.

Embodiment 15. The method of embodiment 11, wherein the candidate selection step comprises a filtering step and an analyzing step.

Embodiment 16. The method of embodiment 11, wherein the candidate selection step comprises applying sequence embeddings from pretrained protein sequence encoders into a K-NN search process and a MIC identification process.

Embodiment 17. The method of embodiment 16, wherein the candidate selection step further comprises selecting an intersection of two potential subsets respectively generated from the K-NN search process and the MIC identification process for post-processing.

Embodiment 18. The method of embodiment 16, wherein the candidate selection step further comprises performing a length selection method, a folding method, a sequence similarity search method, and a structure alignment method.

Embodiment 19. The method of embodiment 1, wherein the running pretrained protein large language models comprise collecting data from multiple open-source antimicrobial peptides (AMPs) databases, wherein the collected data comprise AMPs, wherein the AMPs comprise AMPs reported MIC values and activities selected from the group consisting of antimicrobial, antibacterial and antifungal.

Embodiment 20. The method of embodiment 9, wherein the running pretrained protein large language models comprise filtering the data according to length i100 and instances with redundancy, wherein the instances are split into training, validating, and testing datasets, and wherein the training, validating, and testing datasets are distributed in a ratio of 8:1:1.

Embodiment 21. The method of embodiment 19, wherein the AMPs comprise sub-families, wherein each sub-family has a functional label.

Embodiment 22. The method of embodiment 12, wherein the supervised training comprises rank-based adapters, wherein the rank-based adapters are configured to regulate protein residues interactions for conditional generation of a peptide having functions similar to an input set of training data.

Embodiment 23. The method of embodiment 22, wherein the regulation of protein residues interactions is combined with a special property generation including one, any or all of hydrophobicity, electric charge, cell toxicity, and hemolytic activity, and wherein the combination the regulation of protein residues interactions and the special property generation is utilized to generate a peptide with desired functions for biological applications.

Embodiment 24. The method of embodiment 12, wherein the supervised training is configured to generate fine-tuning peptide samples.

Embodiment 25. The method of embodiment 16, wherein the sequence embeddings comprise data capturing a multiplicity of connections among functional protein sequences.

Embodiment 26. The method of embodiment 17, wherein the K-NN search process comprises:
filtering a generated peptide sample;
assessing similarity between the generated peptide sample and fine-tuning peptide samples; and
computing a distance between the sequence embeddings of the generated peptide sample and the fine-tuning peptide samples;
wherein the sequence embeddings are obtained from the pretrained protein large language models,
wherein the distance between the sequence embeddings is measured utilizing a filtering criterion,
wherein the filtering criterion is computed by weighing average of distances between a sequence embedding of the generated samples and centers of target functional sample sequence embedding, and
wherein a high similarity between the generated peptide sample and the fine-tuning peptide samples correlates with a higher probability that the generated peptide sample possesses similar properties to the fine-tuning peptide sample

Embodiment 27. The method of embodiment 26, wherein the assessing the similarity between the generated peptide sample and the fine-tuning peptide samples comprises assessing based on multiple additional metrics, wherein the multiple additional metrics are selected from the group consisting of Euclidean Distance, Manhattan Distance, and cosine similarity, wherein the multiple additional metrics are normalized and summed together.

Embodiment 28. The method of embodiment 26, wherein the MIC identification process comprises:
entering the sequence embeddings of a candidate peptide;
predicting likelihood that the candidate peptide possesses antibacterial activity based on the sequence embeddings of the candidate peptide;
comparing the predicted MIC of the candidate peptide to a MIC threshold value;
retaining a candidate peptide having a predicted MIC value greater than the MIC threshold value; and
filtering out candidate peptides having a predicted MIC value smaller than the MIC threshold value.

Embodiment 29. The method of embodiment 17, wherein the post-processing comprises: selecting a candidate peptide whose length is equal or less than 50 amino acids.

Embodiment 30. The method of embodiment 18, wherein the performing a folding method comprises simulating a folding process of the candidate peptide; analyzing resulting structure; predicting stability and conformational features of the candidate peptide; and guiding design of a peptide with improved structural integrity and functional properties.

Embodiment 31. The method of embodiment 18, wherein the performing a sequence similarity search comprises: comparing a sequence of the candidate peptide to existing peptide sequences; assessing novelty of the candidate peptide; and assessing similarity of functional domains of the candidate peptide to known functional domains.

Embodiment 32. The method of embodiment 18, wherein the performing a structure alignment comprises utilizing a structure search tool to identify structurally similar peptide families and potential functional peptide domains that possess antimicrobial activity.

The subject invention pertains to a functional protein sequence design that aims to generate novel proteins with human-desired functions. More specifically, the subject invention describes an end-to-end pipeline for functional protein sequence generation. Given a small set of functional data with specific functions, the subject invention can employ a novel protein language model fine-tuning technique, adapting it to protein and training it with large language models. This enables the generation of functionally similar peptide sequences.

The two-tiered improvement method based on autoregressive protein language models is provided. In some embodiments, the subject invention utilizes ProGen2 (arXiv, Ithaca, USA) [29] as the baseline model and implements efficient fine-tuning to explore effective generation space for functional AMPs with desired biochemistry properties. The fine-tuning process allows the models to search for peptides beyond the limitations of the training data, enabling exploration of the vast protein universe and increasing the chances of identifying novel antimicrobial peptides and equipping the generated peptides with specific molecule-based properties [31, 32]. In some embodiments, the subject invention then complements this method with a machine learning-based filtering process, creating a comprehensive end-to-end pipeline. In multi-level filtering stage, cross-validation selection is implemented on candidates from multiple filtering algorithms. Then, a combination of bioinformatics analysis is applied for evaluating the validity and related domain-specific connections of desired instances. The improvements not only achieve the ensemble of multiple desired properties and functions, but also significantly enhance the pLM's ability to generate valid and diverse short peptides.

In some embodiments, the subject invention describes an end-to-end pipeline for functional protein sequence generation. Given a small set of functional data with specific functions, the subject invention can employ a novel protein language model fine-tuning technique, adapting it to protein and training it with large language models. This enables the generation of functionally similar peptide sequences. Simultaneously, a set of custom machine learning filtering methods are utilized to efficiently screen the generated protein sequences. Ultimately, this pipeline allows for the generation of functional sequences with specific functional activities when provided with a limited sample dataset.

In comparison to the traditional design methods, deep learning-based design approaches can effectively identify the interactions between amino acids in protein sequences, thereby expressing higher-level features that are relevant to protein functionality. This novel framework represents an advancement in the field of AMP discovery, offering a streamlined approach for automatic peptide design with a high validation rate and multiple desired properties. The incorporation of the deep learning techniques effectively surmounts the limitations associated with the conventional AMP discovery methods. By leveraging the power of ProGen2 and machine learning methods, a robust pipeline not only enhances the generation of AMPs but also ensures their diversity and novelty. Consequently, this work represents a stride towards tackling the pressing global issue of antimicrobial resistance (AMR), while simultaneously paving the path for further groundbreaking research in this critical domain.

Advantageously, the embodiments of the subject invention efficiently fine-tune a large language model using a small amount of data on protein, addressing the weak performance of large language models in generating short functional protein sequences.

When compared to the existing deep learning approaches, the subject invention connects a small amount of data with a pre-trained large language model, resolving the issue of poor generalization of deep learning models trained with limited data.

The subject invention incorporates the filtering method for potential samples, utilizing a machine learning-based cross-validation approach that significantly improves the success rate and interpretability of the screening process.

The subject invention enables automatic end-to-end generation and filtering based on a given small dataset, resulting in the discovery of functionally diverse peptides within protein families that have not been recognized before. Moreover, the generated samples exhibit high novelty and diversity.

In some embodiments, the subject invention provides a framework for peptide generation and design based on protein intelligent computing. Its main objective is to address the efficient design of specific peptide functionalities. The functionalities designed by this invention exhibit high novelty and diversity, coupled with outstanding perplexity. In practical applications, it can be utilized to generate novel proteins with various functionalities and can be applied in the fields of production and medicine. With the assistance of the protein language model and the interpretable peptide sequence screening method, highly active functional proteins can be successfully designed.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

[1] Mookherjee, N., Anderson, M. A., Haagsman, H. P., Davidson, D. J.: Antimicro-bial host defence peptides: functions and clinical potential. Nature reviews Drug discovery 19(5), 311-332 (2020)
[2] Tang, K. W. K., Millar, B. C., Moore, J. E.: Antimicrobial resistance (amr). British Journal of Biomedical Science 80, 11387 (2023)
[3] Oliveira, K. B. S., Leite, M. L., Cunha, V. A., Cunha, N. B., Franco, O. L.: Chal-lenges and advances in antimicrobial peptide development. Drug Discovery Today, 103629 (2023)
[4] Browne, K., Chakraborty, S., Chen, R., Willcox, M. D., Black, D. S., Walsh, W. R., Kumar, N.: A new era of antibiotics: the clinical potential of antimicrobial peptides. International journal of molecular sciences 21(19), 7047 (2020)
[5] Xuan, J., Feng, W., Wang, J., Wang, R., Zhang, B., Bo, L., Chen, Z.-S., Yang, H., Sun, L.: Antimicrobial peptides for combating drug-resistant bacterial infections. Drug Resistance Updates, 100954 (2023)
[6] Mba, I. E., Nweze, E. I.: Focus: Antimicrobial resistance: Antimicrobial peptides therapy: An emerging alternative for treating drug-resistant bacteria. The Yale journal of biology and medicine 95(4), 445 (2022)
[7] Yan, J., Cai, J., Zhang, B., Wang, Y., Wong, D. F., Siu, S. W.: Recent progress in the discovery and design of antimicrobial peptides using traditional machine learning and deep learning. Antibiotics 11(10), 1451 (2022)
[8] Lei, J., Sun, L., Huang, S., Zhu, C., Li, P., He, J., Mackey, V., Coy, D. H., He, Q.: The antimicrobial peptides and their potential clinical applications. American journal of translational research 11(7), 3919 (2019)
[9] Zhang, Q.-Y., Yan, Z.-B., Meng, Y.-M., Hong, X.-Y., Shao, G., Ma, J.-J., Cheng, X.-R., Liu, J., Kang, J., Fu, C.-Y.: Antimicrobial peptides: mechanism of action, activity and clinical potential. Military Medical Research 8, 1-25 (2021)
[10] Anahtar, M. N., Yang, J. H., Kanjilal, S.: Applications of machine learning to the problem of antimicrobial resistance: an emerging model for translational research. Journal of Clinical Microbiology 59(7), 10-1128 (2021)
[11] Wang, C., Garlick, S., Zloh, M.: Deep learning for novel antimicrobial peptide design. Biomolecules 11(3), 471 (2021)
[12] Mishra, B., Wang, G.: Ab initio design of potent anti-mrsa peptides based on database filtering technology. Journal of the American Chemical Society 134(30), 12426-12429 (2012)
[13] Szymczak, P., Szczurek, E.: Artificial intelligence-driven antimicrobial peptide discovery. Current Opinion in Structural Biology 83, 102733 (2023)
[14] Cardoso, M. H., Orozco, R. Q., Rezende, S. B., Rodrigues, G., Oshiro, K. G., C\ddot{ } andido, E. S., Franco, O. L.: Computer-aided design of antimicrobial peptides: are we generating effective drug candidates? Frontiers in microbiology 10, 3097 (2020)
[15] Palmer, N., Maasch, J. R., Torres, M. D., Fuente-Nunez, C.: Molecular dynam-ics for antimicrobial peptide discovery. Infection and Immunity 89(4), 10-1128 (2021)
[16] Fjell, C. D., Hiss, J. A., Hancock, R. E., Schneider, G.: Designing antimicrobial peptides: form follows function. Nature reviews Drug discovery 11(1), 37-51 (2012)
[17] Cao, Q., Ge, C., Wang, X., Harvey, P. J., Zhang, Z., Ma, Y., Wang, X., Jia, X., Mobli, M., Craik, D. J., et al.: Designing antimicrobial peptides using deep learning and molecular dynamic simulations. Briefings in Bioinformatics 24(2), 058 (2023)
[18] Huang, J., Xu, Y., Xue, Y., Huang, Y., Li, X., Chen, X., Xu, Y., Zhang, D., Zhang, P., Zhao, J., et al.: Identification of potent antimicrobial peptides via a machine-learning pipeline that mines the entire space of peptide sequences Nature Biomedical Engineering, 1-14 (2023)
[19] Szymczak, P., Moz\dot{ }ejko, M., Grzegorzek, T., Jurczak, R., Bauer, M., Neubauer, D., Sikora, K., Michalski, M., Sroka, J., Setny, P., et al.: Discovering highly potent antimicrobial peptides with deep generative model hydramp. Nature Communications 14(1), 1453 (2023)
[20] Dean, S. N., Alvarez, J. A. E., Zabetakis, D., Walper, S. A., Malanoski, A. P.: Pep-vae: variational autoencoder framework for antimicrobial peptide generation and activity prediction. Frontiers in microbiology 12, 725727 (2021)
[21] Hasegawa, K., Moriwaki, Y., Terada, T., Wei, C., Shimizu, K.: Feedback-avpgan: Feedback-guided generative adversarial network for generating antiviral peptides. Journal of Bioinformatics and Computational Biology 20(06), 2250026 (2022)
[22] Chen, Q., Yang, C., Xie, Y., Wang, Y., Li, X., Wang, K., Huang, J., Yan, W.: Gm-pep: A high efficiency method to de novo design functional peptide sequences. Journal of Chemical Information and Modeling 62(10), 2617-2629 (2022)
[23] Uniprot: the universal protein knowledgebase in 2023. Nucleic Acids Research 51(D1), 523-531 (2023)
[24] Das, P., Wadhawan, K., Chang, O., Sercu, T., Santos, C. D., Riemer, M., Chenthamarakshan, V., Padhi, I., Mojsilovic, A.: Pepcvae: Semi-supervised targeted design of antimicrobial peptide sequences. arXiv preprint arXiv:1810.07743 (2018)
[25] Van Oort, C. M., Ferrell, J. B., Remington, J. M., Wshah, S., Li, J.: Ampgan v2: machine learning-guided design of antimicrobial peptides. Journal of chemical information and modeling 61(5), 2198-2207 (2021)
[26] Li, G., Iyer, B., Prasath, V. S., Ni, Y., Salomonis, N.: Deepimmuno: deep learning-empowered prediction and generation of immunogenic peptides for t-cell immunity. Briefings in bioinformatics 22(6), 160 (2021)
[27] Rives, A., Meier, J., Sercu, T., Goyal, S., Lin, Z., Liu, J., Guo, D., Ott, M., Zitnick, C. L., Ma, J., et al.: Biological structure and function emerge from scal-ing unsupervised learning to 250 million protein sequences. Proceedings of the National Academy of Sciences 118(15), 2016239118 (2021)
[28] Madani, A., Krause, B., Greene, E. R., Subramanian, S., Mohr, B. P., Holton, J. M., Olmos Jr, J. L., Xiong, C., Sun, Z. Z., Socher, R., et al.: Large language models gen-erate functional protein sequences across diverse families. Nature Biotechnology, 1-8 (2023)
[29] Nijkamp, E., Ruffolo, J. A., Weinstein, E. N., Naik, N., Madani, A.: Progen2: exploring the boundaries of protein language models. Cell Systems 14(11), 968-978 (2023)
[30] Ferruz, N., Schmidt, S., H\ddot{ } ocker, B.: Protgpt2 is a deep unsupervised language model for protein design. Nature communications 13(1), 4348 (2022)
[31] Hu, E. J., Shen, Y., Wallis, P., Allen-Zhu, Z., Li, Y., Wang, S., Wang, L., Chen, W.: Lora: Low-rank adaptation of large language models. arXiv preprint arXiv:2106.09685 (2021)
[32] Ouyang, L., Wu, J., Jiang, X., Almeida, D., Wainwright, C., Mishkin, P., Zhang, C., Agarwal, S., Slama, K., Ray, A., et al.: Training language models to follow instructions with human feedback. Advances in Neural Information Processing Systems 35, 27730-27744 (2022)
[33] Shi, G., Kang, X., Dong, F., Liu, Y., Zhu, N., Hu, Y., Xu, H., Lao, X., Zheng, H.: Dramp 3.0: an enhanced comprehensive data repository of antimicrobial peptides. Nucleic Acids Research 50(D1), 488-496 (2022)
[34] Novkovi\acute{ } c, M., Simuni\acute{ } c, J., Bojovi\acute{ } c, V., Tossi, A., Jureti\acute{ } c, D.: Dadp: the database of anuran defense peptides. Bioinformatics 28(10), 1406-1407 (2012)
[35] Ye, G., Wu, H., Huang, J., Wang, W., Ge, K., Li, G., Zhong, J., Huang, Q.: Lamp2: a major update of the database linking antimicrobial peptides. Database 2020, 061 (2020)
[36] Jhong, J.-H., Yao, L., Pang, Y., Li, Z., Chung, C.-R., Wang, R., Li, S., Li, W., Luo, M., Ma, R., et al.: dbamp 2.0: updated resource for antimicrobial peptides with an enhanced scanning method for genomic and proteomic data. Nucleic Acids Research 50(D1), 460-470 (2022)
[37] Veltri, D., Kamath, U., Shehu, A.: Deep learning improves antimicrobial peptide recognition. Bioinformatics 34(16), 2740-2747 (2018)
[38] Brown, T., Mann, B., Ryder, N., Subbiah, M., Kaplan, J. D., Dhariwal, P., Neelakantan, A., Shyam, P., Sastry, G., Askell, A., et al.: Language models are few-shot learners. Advances in neural information processing systems 33, 1877-1901 (2020)
[39] Lyu, Z., Yang, P., Lei, J., Zhao, J.: Biological function of antimicrobial peptides on suppressing pathogens and improving host immunity. Antibiotics 12(6), 1037 (2023)
[40] Whisstock, J. C., Lesk, A. M.: Prediction of protein function from protein sequence and structure. Quarterly reviews of biophysics 36(3), 307-340 (2003)
[41] Devlin, J., Chang, M.-W., Lee, K., Toutanova, K.: Bert: Pre-training of deep bidirectional transformers for language understanding. arXiv preprint arXiv:1810.04805 (2018)
[42] Jumper, J., Evans, R., Pritzel, A., Green, T., Figurnov, M., Ronneberger, O., 20 Tunyasuvunakool, K., Bates, R., Z ̌ιdek, A., Potapenko, A., et al.: Highly accurate protein structure prediction with alphafold. Nature 596(7873), 583-589 (2021)
[43] Lin, Z., Akin, H., Rao, R., Hie, B., Zhu, Z., Lu, W., Smetanin, N., Verkuil, R., Kabeli, O., Shmueli, Y., et al.: Evolutionary-scale prediction of atomic-level protein structure with a language model. Science 379(6637), 1123-1130 (2023)
[44] Altschul, S. F., Madden, T. L., Sch\ddot{ } affer, A. A., Zhang, J., Zhang, Z., Miller, W., Lipman, D. J.: Gapped blast and psi-blast: a new generation of protein database search programs. Nucleic acids research 25(17), 3389-3402 (1997)
[45] Kempen, M., Kim, S. S., Tumescheit, C., Mirdita, M., Lee, J., Gilchrist, C. L., S\ddot{ } oding, J., Steinegger, M.: Fast and accurate protein structure search with foldseek. Nature Biotechnology, 1-4 (2023)

We claim:

1. A method based on deep learning-based framework for functional antimicrobial peptide design, the method comprising:
running pretrained protein large language models as a generator; and
enhancing a sample candidate.

2. The method of claim 1, wherein the enhancing a sample candidate comprises performing an automatic pipeline based on machine learning methods and a plurality of bioinformatics methods.

3. The method of claim 2, wherein the automatic pipeline is configured to perform protein inverse folding and computational protein sequence designing.

4. The method of claim 3, wherein the computational protein sequence designing comprises:
running a pretrained deep learning-based protein structure model;
running an autoregressive pretrained protein sequence model; and
running a deep learning-based alignment model.

5. The method of claim 4, wherein the pretrained deep learning-based protein structure model comprises a plurality of data.

6. The method of claim 4, wherein the pretrained deep learning-based protein structure model is configured to learn three-dimensional structures of the proteins.

7. The method of claim 4, wherein the pretrained deep learning-based protein structure model is configured to output latent structure embeddings in a high dimensional space.

8. The method of claim 4, wherein the autoregressive pretrained protein sequence model comprises a protein language model to automatically generate protein sequences.

9. The method of claim 8, wherein the autoregressive pretrained protein sequence model is trained to predict next amino acid in a protein sequence based on a preceding sequence(s) of amino acids.

10. The method of claim 4, wherein the deep learning-based alignment model is trained to learn connections between latent representations of protein sequences and protein structures.

11. A method based on deep learning-based framework for functional antimicrobial peptide design, the method comprising:
an adapter-based step; and
a candidate selection step.

12. The method of claim 11, wherein the adapter-based step comprises a supervised training performed on a plurality of peptide families.

13. The method of claim 12, wherein the adapter-based step further comprises a feedback tuning step configuring the pretrained protein language model for special property generation.

14. The method of claim 13, wherein the special property generation includes one, any, or all of hydrophobicity, electric charge, cell toxicity, and hemolytic activity.

15. The method of claim 14, wherein the candidate selection step comprises a filtering step and an analyzing step.

16. The method of claim 11, wherein the candidate selection step comprises applying sequence embeddings from pretrained protein sequence encoders into a k-nearest neighbors (K-NN) search process and a minimum inhibitory concentration (MIC) identification process.

17. The method of claim 16, wherein the candidate selection step further comprises selecting an intersection of two potential subsets respectively generated from the K-NN search process and the MIC identification process for post-processing.

18. The method of claim 16, wherein the candidate selection step further comprises performing a length selection method, a folding method, a sequence similarity search method, and a structure alignment method.

19. The method of claim 1, wherein the running pretrained protein large language models comprises collecting data from multiple open-source antimicrobial peptides (AMPs) databases, wherein the collected data comprise AMPs, wherein the AMPs comprise AMPs reported MIC values and activities selected from the group consisting of antimicrobial, antibacterial, and antifungal.

20. The method of claim 19, wherein the running pretrained protein large language models comprises filtering the data according to length i100 and instances with redundancy, wherein the instances are split into training, validating, and testing datasets, and wherein the training, validating, and testing datasets are distributed in a ratio of 8:1:1.

21. The method of claim 19, wherein the AMPs comprise sub-families, and wherein each sub-family has a functional label.

22. The method of claim 12, wherein the supervised training comprises rank-based adapters, and wherein the rank-based adapters are configured to regulate protein residues interactions for conditional generation of a peptide having functions similar to an input set of training data.

23. The method of claim 22, wherein the regulation of protein residues interactions is combined with a special property generation including one, any, or all of hydrophobicity, electric charge, cell toxicity, and hemolytic activity, and wherein the combination of the regulation of protein residues interactions and the special property generation is utilized to generate a peptide with desired functions for biological applications.

24. The method of claim 12, wherein the supervised training is configured to generate fine-tuning peptide samples.

25. The method of claim 16, wherein the sequence embeddings comprise data capturing a multiplicity of connections among functional protein sequences.

26. The method of claim 17, wherein the K-NN search process comprises:
filtering a generated peptide sample;
assessing similarity between the generated peptide sample and fine-tuning peptide samples; and
computing a distance between the sequence embeddings of the generated peptide sample and the fine-tuning peptide samples;
wherein the sequence embeddings are obtained from the pretrained protein large language models,
wherein the distance between the sequence embeddings is measured utilizing a filtering criterion,
wherein the filtering criterion is computed by weighing average of distances between a sequence embedding of the generated samples and centers of target functional sample sequence embedding, and
wherein a high similarity between the generated peptide sample and the fine-tuning peptide samples correlates with a higher probability that the generated peptide sample possesses similar properties to the fine-tuning peptide samples.

27. The method of claim 26, wherein the assessing the similarity between the generated peptide sample and the fine-tuning peptide samples comprises assessing based on multiple additional metrics, wherein the multiple additional metrics are selected from the group consisting of Euclidean Distance, Manhattan Distance, and cosine similarity, wherein the multiple additional metrics are normalized and summed together.

28. The method of claim 26, wherein the MIC identification process comprises:
entering the sequence embeddings of a candidate peptide;
predicting a likelihood that the candidate peptide possesses antibacterial activity based on the sequence embeddings of the candidate peptide;
comparing the predicted MIC of the candidate peptide to a MIC threshold value;
retaining a candidate peptide having a predicted MIC value greater than the MIC threshold value; and
filtering out candidate peptides having a predicted MIC value smaller than the MIC threshold value.

29. The method of claim 17, wherein the post-processing comprises: selecting a candidate peptide whose length is equal or less than 50 amino acids.

30. The method of claim 18, wherein the performing a folding method comprises simulating a folding process of the candidate peptide; analyzing resulting structure; predicting stability and conformational features of the candidate peptide; and guiding design of a peptide with improved structural integrity and functional properties.

31. The method of claim 18, wherein the performing a sequence similarity search comprises: comparing a sequence of the candidate peptide to existing peptide sequences; assessing novelty of the candidate peptide; and assessing similarity of functional domains of the candidate peptide to known functional domains.

32. The method of claim 18, wherein the performing a structure alignment comprises utilizing a structure search tool to identify structurally similar peptide families and potential functional peptide domains that possess antimicrobial activity.
